(19) United States
(12) Patent Application Publication
(10) Pub. No.: US 2010/0273750 A1
(43) Pub. Date: Oct. 28, 2010

(54) SEROTONIN RECEPTOR ANTAGONISTS FOR TREATING ARTHRITIS

(75) Inventors: Brian M. Foxwell, London (GB); Sandra Sacre, London (GB)

Correspondence Address: KAREN S. CANADY;CANADY & LORTZ LLP, 4201 Wilshire BLV Suite 622, LOS ANGELES, CA, 90010, US

(73) Assignee: IMPERIAL INNOVATIONS LIMITED, London (GB)

(21) Appl. No.: 12/524,141

(22) PCT Filed: Jan. 22, 2008
(86) PCT No.: PCT/GB08/00222

§ 371 Date: Apr. 1, 2010

(30) Foreign Application Priority Data
Jan. 22, 2007 (GB) 0701170.3

(51) Int. Cl. A61K 31/56; A61K 31/55; A61K 31/5415; A61K 31/4178; A61K 31/553; A61K 31/519; A61K 31/496; A61K 31/437; A61K 31/517; A61P 19/02; A61P 17/06; A61P 29/00; A61P 31/00

(52) U.S. Cl. 514/171; 514/214.02; 514/226.2; 514/397; 514/211.13; 514/259.2; 514/252.13; 514/288; 514/266.21

(57) ABSTRACT

The present invention relates to serotonin receptor antagonists for treating inflammatory arthritis and related conditions. The present invention also relates to a kit comprising a pharmaceutical composition comprising a serotonin receptor antagonist and instructions indicating that the composition is for use in treating inflammatory arthritis and related conditions. In addition, the present invention relates to a method of treating inflammatory arthritis and related conditions comprising administration of a serotonin receptor antagonist. The method is preferably carried out on a subject in need of treatment. The inflammatory arthritis is preferably rheumatoid arthritis.

The present invention relates to serotonin receptor antagonists for treating inflammatory arthritis and related conditions. The present invention also relates to a kit comprising a pharmaceutical composition comprising a serotonin receptor antagonist and instructions indicating that the composition is for use in treating inflammatory arthritis and related conditions. In addition, the present invention relates to a method of treating inflammatory arthritis and related conditions comprising administration of a serotonin receptor antagonist. The method is preferably carried out on a subject in need of treatment. The inflammatory arthritis is preferably rheumatoid arthritis.

Rheumatoid arthritis is a chronic inflammatory disease occurring in 0.5%-1% of the population. This disease affects the joints and is characterized by thickening of the synovial membrane. This disease leads to progressive debilitation in joint function which results in pain, disability, loss of man power and shorter life expectancy. In addition there are a number of similar related conditions; psoriatic arthritis, juvenile arthritis, ankylosing spondelytis, Crohn's disease and psoriasis.

Current treatments for rheumatoid arthritis have a number of disadvantages, including expense and/or severe side effects. At present, steroids such as dexamethasone and methyl-prednisolone, are widely used in the treatment of rheumatoid arthritis. While treatment with steroids can be effective, there are a number of serious side effects. These side effects include hypertension, growth deficiencies in younger patients, osteoporosis, cataracts, psychosis, elevated blood sugar, glaucoma, etc. In addition, some patients are resistant to long-term use of steroids or become so with time.

New and alternative treatments currently used for rheumatoid arthritis are based on biologicals such as antibodies and soluble receptors. The most widely used of these is based on blocking TNF function with neutralizing antibodies or soluble receptors and has defined TNF as a key pathway in the inflammatory processes in rheumatoid arthritis. This type of anti-TNF therapy has been successful in the treatment of a number of diseases, with a substantial proportion of patients (approximately 60%) showing significant clinical benefit. Besides treating the inflammation, anti-TNF therapy also prevents joint destruction and thus is considered to have disease modifying anti-arthritic drug (DMARD) activity. However, it is extremely expensive and this places a heavy financial burden either on the patient or the healthcare system or both and this seriously limits availability. Many patients in the developed world and the majority in the developing world are not able to afford this treatment. In addition, possible side effects of anti-TNF therapy include anaphylaxis and cytopenia. Moreover systemic neutralisation of TNF leads to increased susceptibility to infection and the long-term effects are still unknown. Currently it is not possible to take anti-TNF drugs orally, which is a disadvantage. Moreover of those patients that do respond approximately 50% will become refractory to treatment in 2 years due to the development of immunity to the biological treatment.

Another class of drugs are the disease modifying anti-rheumatic drugs (DMARDS). An example of these is methotrexate, an anti-metabolite drug, which is widely used for the treatment of rheumatoid arthritis, psoriatic arthritis and psoriasis. Methotrexate has been successful in the treatment of these diseases, but can cause substantial side effects, such as severe skin reaction, infections such as pneumonia, severe damage to liver, kidneys, lungs and gastrointestinal tract. Again a significant number of patients do not respond or become refractory with time.

A number of DMARD pharmaceutical agents containing gold are also used in the treatment of rheumatoid arthritis. Examples of such agents include gold sodium thiomalate and auranofin. Potential side effects from being treated with anti-inflammatory gold agents are oral ulcers, altered taste, serious skin rashes, renal problems, inflammation of the intestines (enterocolitis), liver injury and lung disease. Furthermore, resistance to gold has been known to develop in patients.

A further class of drugs are the non-steroidal anti-inflammatory drugs (NSAID's). These are used to alleviate symptoms but not modify disease progression and includes the Cox 2 inhibitors “VIOXX”®, (a registered trademark of Merck & Co., Inc) and “CELEBREX”®, (a registered trademark of G.D. Searle & Co). However there is major concern about the safety of these drugs with evidence of increased cardiovascular risk.

As a result of lack of efficacy, development of resistance, unacceptable side-effects and expense of existing treatments and route of administration, it is hugely desirable to find alternative treatments for rheumatoid arthritis. Thus there is a huge unmet medical need for an orally-available, well tolerated, inexpensive drug that could selectively block the production of TNF associated with pathological inflammation found in RA and related conditions.

Serotonin receptor antagonists are well known for use in the treatment of depression, nausea and psychotic disorders. Past work in this area has indicated that at least one serotonin receptor antagonist, mianserine, actually increases inflammatory arthritis (Letter to the editor of the British Journal of Rheumatology, 1991).

The first aspect of the present invention provides a serotonin receptor antagonist for treating inflammatory arthritis and related conditions.

The inflammatory arthritis is anti-TNF responsive arthritis, as are the related conditions psoriatic arthritis, juvenile arthritis, ankylosing spondelytis, Crohn's disease and psoriasis. These drugs are envisaged to be useful in diseases where treatment with anti-TNF has shown to be beneficial e.g. Crohn's disease and psoriasis, but not useful in diseases where anti-TNF has been shown to be ineffective or to exacerbate disease, e.g. systemic lupus erythematosus, multiple sclerosis and ulcerative colitis. Mianserine is unable to inhibit LPS induced TNF indicating that mianserine does not possess general anti-inflammatory properties and thus would not be expected to be beneficial in the treatment of sepsis where TNF is produced by LPS stimulation.

In a preferred embodiment the inflammatory arthritis is rheumatoid arthritis and the related conditions are psoriatic arthritis, juvenile arthritis, ankylosing spondelytis, Crohn's disease and psoriasis.

A serotonin receptor antagonist of the invention is any compound that inhibits a member of the serotonin receptor family. This includes serotonin receptor inverse agonists.

As is known in the art, a serotonin receptor antagonist is a compound that prevents the actions of serotonin (5-hydroxytryptamine or 5-HT) by binding to a serotonin receptor. Serotonin receptor antagonists include inverse agonists. A serotonin receptor inverse agonist is a drug which binds to a serotonin receptor producing an antagonistic effect but additionally exerts a different pharmacological effect to serotonin.

A serotonin receptor antagonist of the invention inhibits TNF production.

Information on determining serotonin receptor antagonism can be found in Cohen et al., J. Pharmacol. Exp. Ther. (1988) 244 (1): 106.

5-HT receptors are receptors for the neurotransmitter and peripheral signal mediator serotonin, also known as 5-hydroxytryptamine or 5-HT. Serotonin (5-HT) receptors are divided into seven distinct classes 5-HT₁-5-HT₇ comprising at least 14 distinct members. It is through these receptors that serotonin produces its effects.

In one embodiment, the serotonin receptor antagonist is a 5-HT₁, 5-HT₂, 5-HT₃, 5-HT₄, 5-HT₅, 5-HT₆ or a 5-HT₇ receptor antagonist.

Compounds of the invention can be promazine (including promazine hydrochloride), ketanserine (including ketanserine tartrate), amoxapine, maprotiline, mianserine (including mianserine hydrochloride), mirtazapine, nefazodone, setiptiline, trazodone, ritanserin, spiperone, methiothepin, GR55562, SB 224289, chloropromazine, clozapine, haloperidol, loxapine, risperidone, mesulergine, cyproheptadine, ML 10375, olanzapine, WAY 100635, GR113808, memantine, metoclopramide, renzapride, ondansetron, alosetron, agomelatine, pizotifen, metergoline, ergotamine, cyamemazine, BRL43694A, RS100235, Ro630563, SB271046, SB357134, SB258719, SB269970, cyamemazine, SB242084, RS102221, granisetron, SB204070, tropisetron, SB236057, BRL15572, MDL100907, SB200646, SB204741, metitepine, ricasetron, SR46349-B, ICS 205-930, clotiapine.

The use may be of combinations of two or more serotonin receptor antagonists, which may be for simultaneous, separate or sequential use.

The serotonin receptor antagonists may be used in combination with a further anti-inflammatory agent. Administration of the serotonin receptor antagonists and other anti-inflammatory agent can be simultaneous, separate and/or sequential. The serotonin receptor antagonists, in combination with another pharmaceutical agent, can act additively or synergistically.

The other anti-inflammatory agent may be termed a non-steroidal anti-inflammatory agent (NSAID), a disease modifying anti-rheumatic drug (DMARD), a biological agent (biologicals), a statin (including HMG-CoA reductase inhibitors such as simvastatin), a steroid, an immunosuppressive agent, a salicylate and/or a microbicidal agent. Non-steroidal anti-inflammatory agents include anti-metabolite agents (including methotrexate) and anti-inflammatory gold agents (including gold sodium thiomalate, aurothiomalate or gold salts, such as auranofin). Biologicals include anti-TNF agents (including adalimumab, etanercept, infliximab, anti-IL-1 reagents, anti-IL-6 reagents, anti-B cell reagents (retoximab), anti-T cell reagents (anti-CD4 antibodies), anti-IL-15 reagents, anti-CLTA4 reagents, anti-RAGE reagents), antibodies, soluble receptors, receptor binding proteins, cytokine binding proteins, mutant proteins with altered or attenuated functions, RNAi, polynucleotide aptemers, antisense oligonucleotides or omega 3 fatty acids. Steroids include cortisone, prednisolone or dexamethasone. Immunosuppressive agents include cylcosporin, FK506, rapamycin, mycophenolic acid. Salicylates include aspirin, sodium salicylate, choline salicylate and magnesium salicylate. Microbicidal agents include quinine and chloroquine. For example, the serotonin receptor antagonist may be administered in combination with one or more of an NSAID, DMARD, or immunosuppressant.

The further anti-inflammatory agent is administered by any appropriate route, for example oral (including buccal or sublingual), suppositories, topical (including buccal, sublingual or transdermal), or parenteral (including subcutaneous, intramuscular, intravenous or intradermal) route. Where the further anti-inflammatory agent is administered orally, it may be administered as part of the same composition as the serotonin receptor antagonists.

The serotonin receptor antagonists of the present invention are those serotonin receptor antagonists which are non-toxic to living cells.

The present invention also provides the use of a serotonin receptor antagonist in the manufacture of a medicament for treating inflammatory arthritis and related conditions.

The second aspect is a kit comprising a pharmaceutical composition comprising a serotonin receptor antagonist and instructions indicating that the composition is for use in treating inflammatory arthritis and related conditions.

Compositions in accordance with the invention may be supplied as part of a sterile, pharmaceutical composition which will normally include a pharmaceutically acceptable carrier. This pharmaceutical composition may be in any suitable form. It may be provided in unit dosage form and will generally be provided in a sealed container. The kit of the invention may comprise a plurality of said unit dosage forms.

The oral pharmaceutical compositions may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc.

For the preparation of solutions and syrups, excipients which may be used include for example water, polyols and sugars. For the preparation of suspensions oils (e.g. vegetable oils) may be used to provide oil-in-water or water in oil suspensions.

The pharmaceutical compositions may contain preserving agents, solubilising agents, stabilising agents, wetting agents, emulsifiers, sweeteners, colourants, odourants, salts, buffers, coating agents or antioxidants. They may also contain further therapeutically active agents.

Route of administration may include; parenterally (including subcutaneous, intramuscular, intravenous, by means of, for example a drip patch), some further suitable routes of administration include (but are not limited to) oral (including buccal and sublingual), rectal, nasal, topical, infusion, vaginal, intradermal, intraperitoneally, intracranially, intrathecal and epidural administration or administration via oral or nasal inhalation, by means of, for example a nebuliser or inhaler, or by an implant.

For administration via the oral or nasal inhalation routes the may be delivered using a mechanical form including, but not restricted to, an inhaler or nebuliser device.

Further, where the oral or nasal inhalation routes are used, administration is by a SPAG (small particulate aerosol generator) may be used.

Dosages of the substances of the present invention can vary between wide limits, depending upon the condition to be treated, the health of the individual to be treated, etc. and a physician may determine appropriate dosages to be used. The dosage may be repeated as often as appropriate. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

The compositions and uses described in this application are envisaged to have human, animal and veterinary applications. They are preferably applicable to mammals, in particular humans but are also applicable for use in production animals, in particular sheep, cows, pigs, chickens and goats, as well as companion animals, in particular cats and dogs and sporting animals, such as horses.

The third aspect of the invention is a method of treating inflammatory arthritis and related conditions comprising administration of a serotonin receptor antagonist. In the third aspect of the invention, the method is preferably carried out on a subject in need of treatment.

In the present invention, the term “treatment” includes prophylactic treatment (i.e. prevention). In most circumstances, prevention of inflammatory arthritis or a related condition is unlikely to be carried out. Usually, it is only when the presence of inflammatory arthritis or a related condition is diagnosed in a subject that prevention means are applied. However, prophylactic treatment may be appropriate if there is a known family history of significant inflammatory arthritis or a related condition or if tests (e.g. genetic tests) identify that an individual has a predisposition to inflammatory arthritis or a related condition.

The preferred embodiments, as described for the first aspect of the invention, are the same for other aspects of the invention mutatis mutandis.

The present invention is described with references to the drawings, in which:

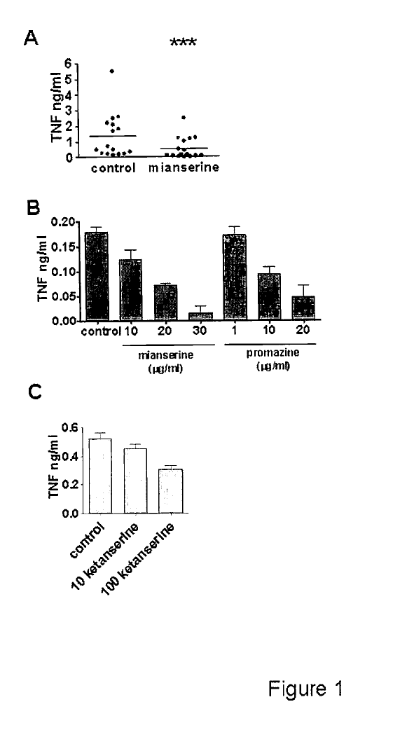

FIG. 1 illustrates inhibition of spontaneous cytokine release from RA synovial membrane cultures by mianserine hydrochloride, promazine hydrochloride and ketanserine tartrate. Graph (A) illustrates inhibition of TNF levels (n=16) by 30 μg/ml mianserine hydrochloride as measured by ELISA. Each point represents an individual donor. Graph (B) illustrates a dose dependent inhibition of TNF by mianserine hydrochloride and promazine hydrochloride. Graph (C) illustrates dose dependent inhibition of TNF in RA synovial membrane cultures treated with 10 μM or 100 μM ketanserine tartrate. ***=P<0.001

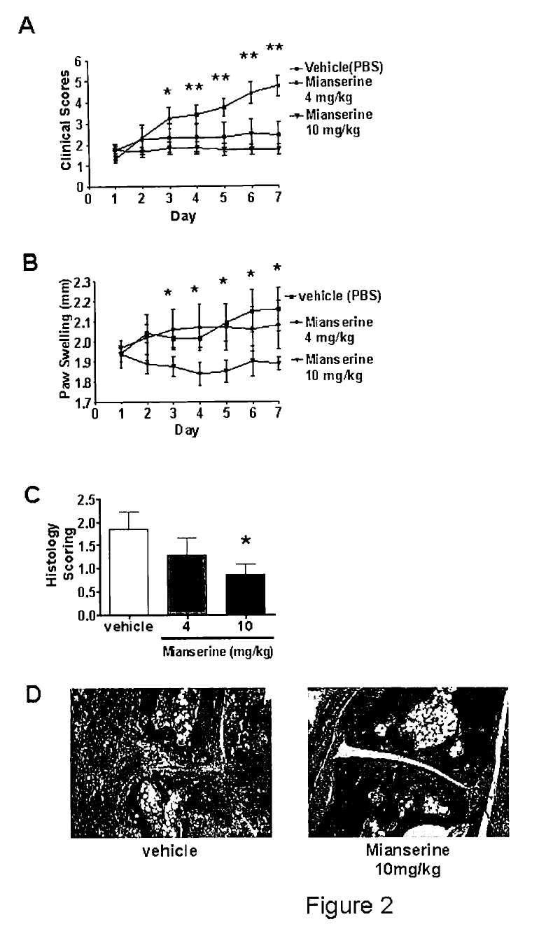

FIG. 2 illustrates inhibition of disease progression in the CIA model of rheumatoid arthritis by mianserine hydrochloride. Graph (A) illustrates clinical score, Graph (B) illustrates paw swelling, Graph (C) illustrates histology scoring and Graph (D) illustrates histology images of a joint. The statistics were calculated using a Mann-Whitney one tailed test **=P<0.01, *=P<0.05, n=7, for the 10 mg/kg group versus the vehicle control.

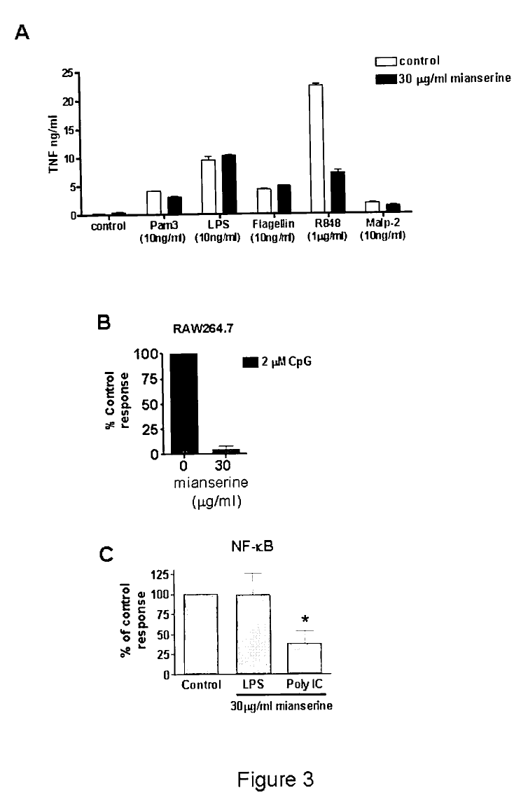

FIG. 3 Graph (A) illustrates TNF production in primary human macrophages stimulated with TLR ligands in media alone or in the presence of 30 μg/ml mianserine hydrochloride. Graph (B) illustrates inhibition by mianserine hydrochloride of TNF production in CpG stimulated murine RAW264.7 macrophages (n=2). Graph (C) illustrates inhibition by mianserine hydrochloride of NF-κB activity activated by the TLR3 ligand poly IC (20 μg/ml) but not by the TLR4 ligand LPS (10 ng/ml) in rheumatoid synovial fibroblasts (n=3), *=P<0.05.

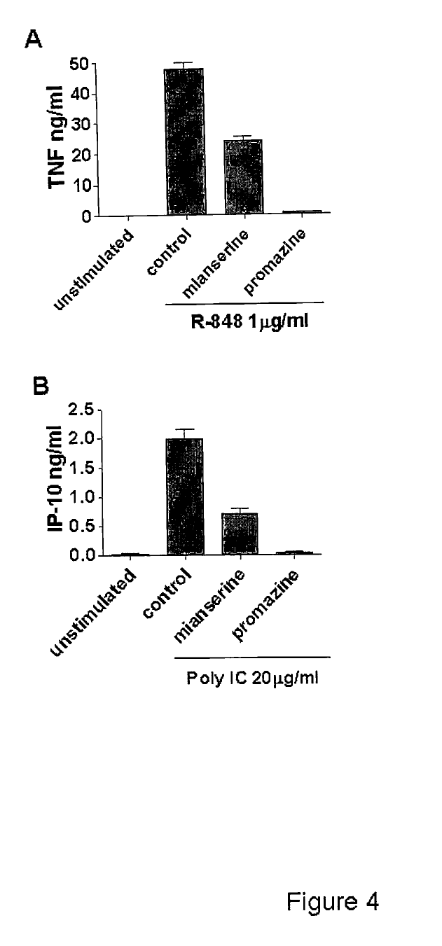

FIG. 4 Graph (A) illustrates TNF production from human macrophages stimulated with 1 μg/ml R848 in the media alone or in the presence of 30 μg/ml mianserine hydrochloride or 30 μg/ml promazine hydrochloride. Graph (B) illustrates IP-10 levels from macrophages stimulated with 20 μg/ml poly IC, in media alone or in the presence of 30 μg/ml mianserine hydrochloride or 30 μg/ml promazine hydrochloride.

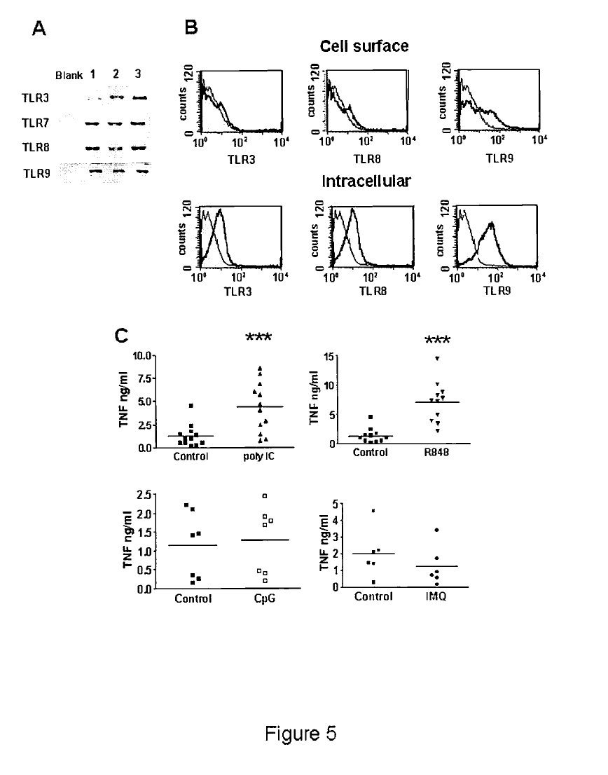

FIG. 5 illustrates that TLR3, 7, 8 and 9 are expressed in the rheumatoid synovial membrane cultures but only TLR3 and 8 induce TNF production. Graph (A) RT-PCR illustrates the presence of TLR3, TLR7, TLR8 and TLR9 mRNA in 3 separate donors. Graph (B) illustrates FACS analysis of cell surface and intracellular expression of TLR3, TLR8 and TLR9. Representative histogram plots of 3 independent donors are shown. Graph (C) illustrates TNF production in rheumatoid membrane synovial cells after stimulation with 20 μg/ml Poly IC (TLR3), 1 μg/ml imiquimod (TLR7), 1 μg/ml R-848 (TLR7/8) and 2 μM CpG (TLR9).

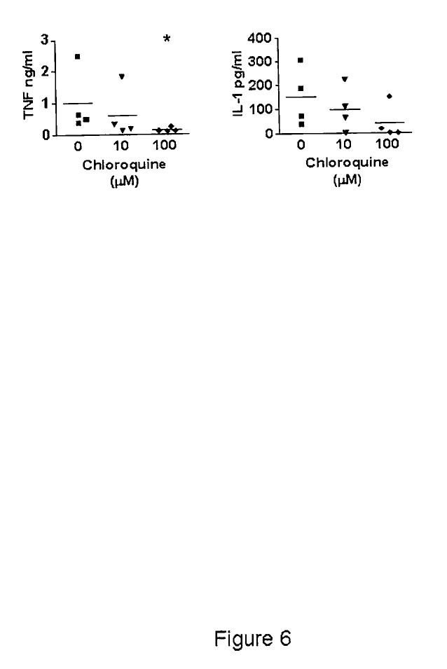

FIG. 6 illustrates the mechanism of inhibition of TNF by mianserine hydrochloride in RA membrane cell may be due to inhibition of the TLRs. Addition of chloroquine to RA membrane cell cultures for 24 hours (n=4). *=P<0.05

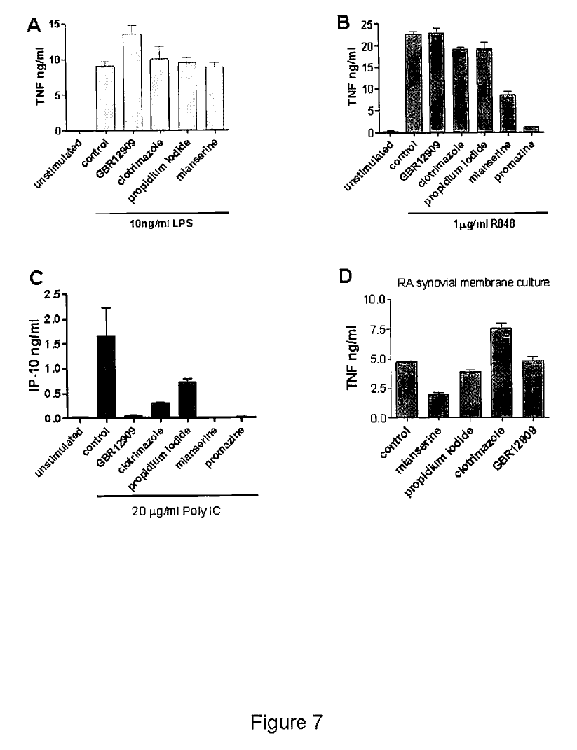

FIG. 7 (A) illustrates that GBR12909, Clotrimazole, Propidium iodide (PI) and mianserine hydrochloride do not inhibit TNF production in macrophages stimulated with LPS. Graph (B) illustrates that only mianserine hydrochloride and promazine hydrochloride inhibit R-848 induced TNF. Graph (C) illustrates that GBR12909, Clotrimazole, Propidium iodide, mianserine hydrochloride and promazine hydrochloride are all able to inhibit Poly IC induced IP-10. Graph (D) illustrates GBR12909, Clotrimazole, Propidium iodide, do not inhibit TNF production in the rheumatoid arthritis synovial membrane culture while mianserine hydrochloride does inhibit.

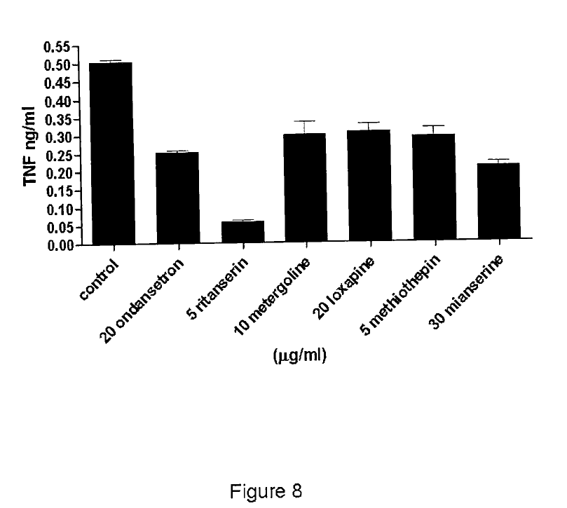

FIG. 8 illustrates that the serotonin receptor antagonists: ondansetron, metergoline, loxapine, ritanserin, methiothepin and mianserine inhibit spontaneous TNF production from human RA synovial membrane cultures, as measured by ELISA.

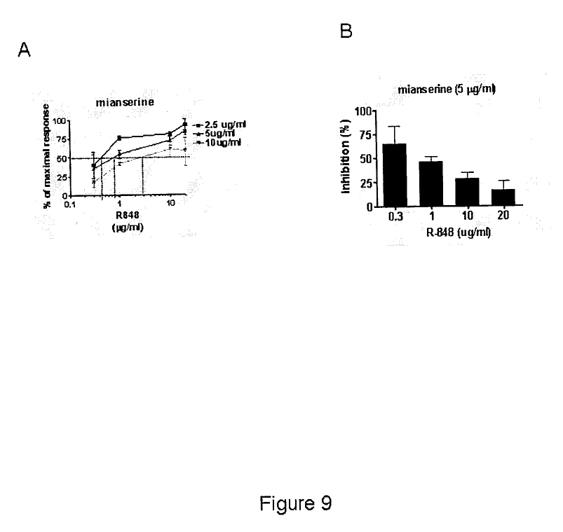

FIG. 9 illustrates competitive inhibition of R-848 by mianserine. Primary human macrophages were incubated with media containing 0.3, 1, 10 or 20 μg/ml R-848 with media alone or in the presence of either (A) 2.5, 5 or 10 μg/ml mianserine or (B) 5 μg/ml mianserine for 6 hours. TNF production was measures by ELISA. Data was pooled from three separate donors. Results are shown as (A) percentage of the maximal TNF response or (B) the percentage inhibition of TNF production.

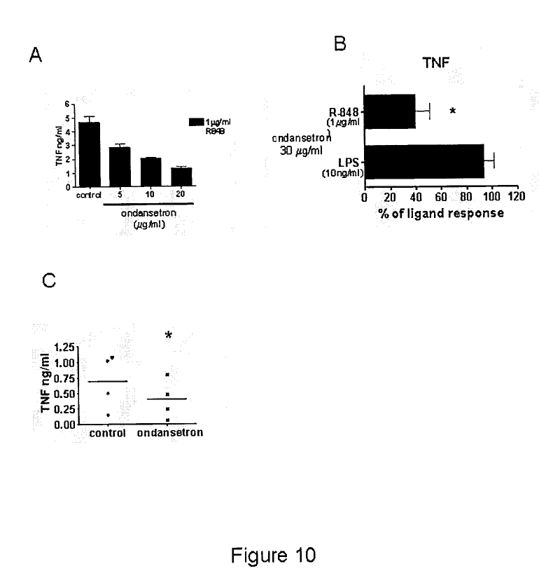

FIG. 10 illustrates inhibition of R-848 induced TNF production from primary human macrophages and spontaneous TNF production from RA cultures by ondansetron. (A) illustrates a dose dependant inhibition of R-848 induced TNF production from human macrophages. (B) illustrates that ondansetron significantly inhibits R-848 induced TNF but not LPS induced TNF in human macrophages. (C) illustrates significant inhibition of spontaneous TNF production from human RA synovial cultures by ondansetron (n=5)*=P<0.05.

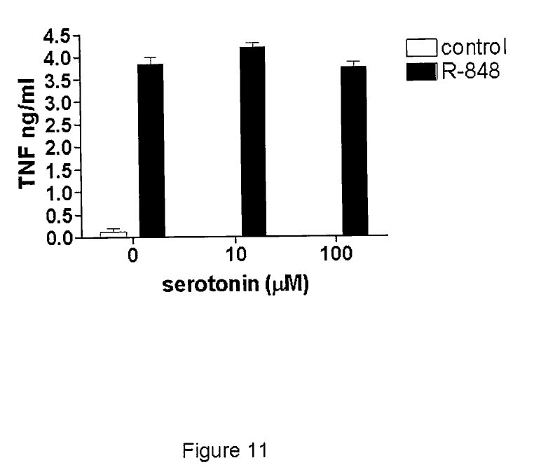

FIG. 11 illustrates that serotonin (5-HT) does not inhibit TNF production induced by R-848 as measured by ELISA.

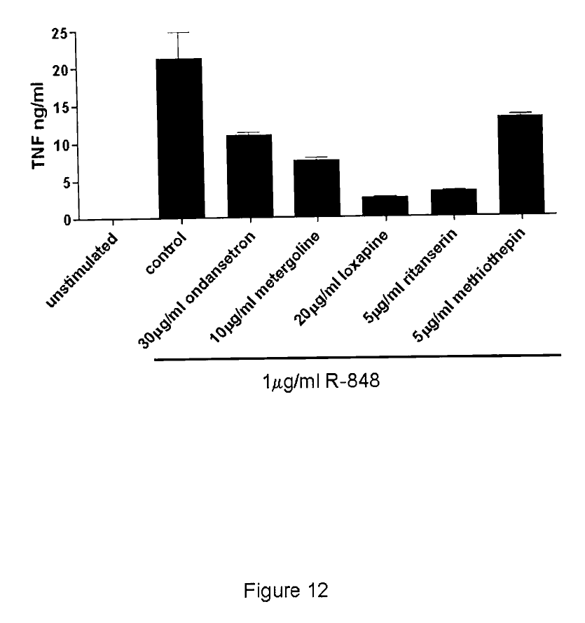

FIG. 12 illustrates that ondansetron, metergoline, loxapine, ritanserin and methiothepin inhibit TNF production induced by R-848 from primary human macrophages, as measured by ELISA.

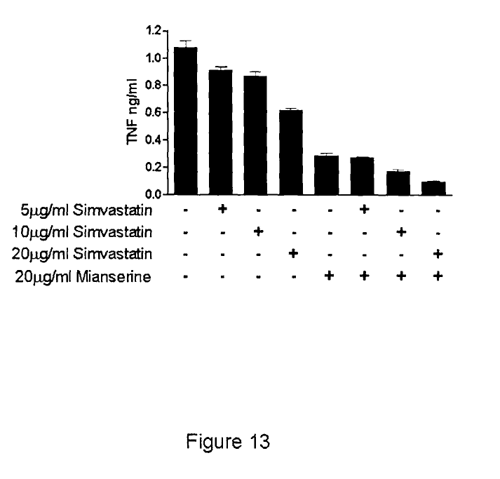

FIG. 13 illustrates that simvastatin and mianserine inhibit spontaneous TNF production from human RA synovial membrane cultures as measured by ELISA and produce an additive effect when added together.

The present invention is described with reference to the following non-limiting examples:

### EXAMPLES

### General Materials and Methods

### Reagents

Cell culture reagents used were Penicillin-Streptomycin, RPMI 1640 and DMEM obtained from Cambrex (Belgium), Indomethacin from Sigma (USA) and FBS from PAA (Austria). The TLR ligands used were chloroform extracted Escherichia coli (E. coli) LPS and resiquimod (R-848), murine CpG (ODN1826), human CpG (ODN 2006) and imiquimod from Invivogen (USA), Macrophage-activating lipopeptide (Malp-2), Flagellin (purified) and were from Alexis (UK). Mianserine hydrochloride was purchased from Sequoia research products (UK). Chloroquine diphosphate salt, Clotrimazole, Propidium iodide (PI), GBR12909 and promazine hydrochloride were purchased from Sigma (USA). Ketanserine tartrate was purchased from Tocris bioscience (UK). Pam₃cys-ser(lys)₄.3HCl (Pam3) was from Alexis (UK). The antibodies used for FACS were FITC conjugated α-TLR3, α-TLR8 and α-TLR9 from Imgenex (USA) and a IgG1-FITC control from BD Pharmingen.

### Reverse Transcriptase-Polymerase Chain Reaction (RT-PCR)

RNA was isolated using the Qiagen RNA Blood isolation kit (Qiagen, Valencia, Calif.) according to the manufacturer's instructions. Total RNA was reverse transcribed with Superscript II RNase H⁻ reverse transcriptase (Gibco Life Technologies, Carlsbad, Calif.) and oligo (dT) primer. For human TLR3 amplification, the primers 5′-GCAAACACAAGCATTCGGAATCT-3′ and 5′-TTGAAGGCTTGGGACCAAGGCA-3′ were used with an annealing temperature of 62° C. For human TLR7 amplification, the primers 5′-TCTACCTGGGCCAAAACTGTT-3′ and 5′-GGCACATGCTGAAGAGAGTTA-3′ were used with an annealing temperature of 60° C. For human TLR8 amplification, the primers 5′-CCGACTTGGAAGTTCTAGATC-3′ and 5′-AATGCTTCATTTGGGATGTGCT-3′ were used with an annealing temperature of 60° C. For human TLR9 amplification, the primers 5′-GTGCCCCACTTCTCCATG-3′ and 5′-GGCACAGTCATGATGTTGTTG-3′ were used with an annealing temperature of 55° C. Amplification was performed in a Dyad PCR machine (MJ Instruments). Subsequent PCR amplification consisted of 35 cycles.

### ELISA (Enzyme-Linked Immunosorbent Assay)

Macrophages or RA synovial membrane cell cultures were stimulated with 10 ng/ml chloroform-extracted LPS, 10 ng/ml Pam3, 10 ng/ml Flagellin, 10 ng/ml Malp-2, 20 μg/ml poly IC, 2 μM CpG (human/mouse) or 1 μg/ml R-848 in complete media for 6 h. Supernatants from RA cultures were harvested after 24 hours. All reagents other than LPS were free from LPS contamination as assessed using the limulus amebocyte lysate (LAL) assay from Cambrex (USA).

Sandwich ELISAs were employed to measure TNF, (Pharmingen, UK). IP-10 (R&D, USA) and IL-1β (Bioscource. USA). Absorbance was read on a spectrophotometric ELISA plate reader (Labsystems Multiscan Biochromic) and analyzed using Ascent software V2.6 (Thermo Labsystems, Cambridge, United Kingdom). All results are expressed as the mean cytokine concentration±SD obtained from triplicate cultures per condition. Cell viability was not significantly affected over this time period when examined by the 3-[4,5 dimethylthiazol-2-yl]-2,5-diphenyl-tetrazolium bromide (MTT) assay (Sigma).

### Luciferase Assay

Synovial fibroblasts were cultured in a 96-well plate, infected with recombinant adenovirus containing a NF-κB Luciferase reporter gene at a MOI of 200:1. The cells were allowed to rest for 24 h prior to stimulation. Cells were preincubated with 30 μg/ml mianserine hydrochloride for 30 minutes, then stimulated with 10 ng/ml LPS or 20 μg/ml Poly IC for 6 h. The cells were then washed once in PBS and lysed with 100 μl of CAT lysis buffer (0.65% (v/v) of Nonidet P-40, 10 mM Tris-HCl pH 8, 0.1 mM EDTA pH 8, 150 mM NaCl). 50 μl of cell lysate were mixed with 120 μl luciferase assay buffer (25 mM Tris-phosphate pH 7.8, 8 mM MgCl₂, 1 mM EDTA, 1% (v/v) Triton X-100, 1% (v/v) glycerol, 1 mM DTT, 0.5 mM ATP) in the well of a luminometer cuvette strip. Luciferase activity was measured with a Luminometer (Thermo Labsystems, U.K.) by adding 300 of luciferin (Bright-Glo luciferase assay system; Promega, Madison, Wis.) per assay point.

### Statistical Methods

Mean, standard deviation (SD) and statistical significance were calculated using GraphPad version 3 (GraphPad Software Inc., USA). For statistical analysis, a one tailed student's t-test of paired data was used with a 95% confidence interval and the Mann-Whitney test.

### Cell Culture

RA synovial membrane cells were isolated from patients undergoing joint replacement surgery as previously described (Foxwell et al., Proc. Natl. Acad. Sci. USA 95, 8211-8215, 1998). All patients gave written informed consent and the study was approved by the local ethics committee. Immediately after isolation, cells were used for mRNA analysis, stained by FACS or cultured at 1×10⁵ cells/well in 96-well tissue culture plates (Falcon, UK) in RPMI 1640 containing 10% (v/v) FBS and 100 U/ml penicillin/streptomycin. Primary human synovial fibroblasts were cultured as described previously (Butler et al. Eur. Cytokine Netw. 5:441-8, 1994). Peripheral blood monocytes were isolated and cultured as previously described (Foxwell et al. Proc. Natl. Acad. Sci. USA 95, 8211-8215, 1998). Macrophages were derived from monocytes after differentiation for 4 days with 100 ng/ml M-CSF (PeproTech, UK). The mouse macrophage cell line RAW 264.7 was maintained in Dulbecco's modified Eagle's medium supplemented with 10% fetal bovine serum.

### Example 1

Rheumatoid arthritis synovial membrane cells were cultured for 24 hours in the presence of media alone or media containing 30 μg/ml mianserine hydrochloride. Supernatants were harvested and levels of cytokines (A) TNF (n=16) were measured by ELISA. Addition of mianserine hydrochloride at the time of culture led to a significant decrease in the levels of TNF (59%±13, p=0.0007) (FIG. 1, Graph A). A students paired t-test was used to analyse the data.

Rheumatoid arthritis synovial membrane cells were also cultured in the presence of media alone or media containing 10 μg/ml, 20 μg/ml and 30 μg/ml mianserine hydrochloride or promazine hydrochloride and levels of TNF were measured. TNF activity was dose dependently inhibited in cells treated with mianserine hydrochloride and promazine hydrochloride (see FIG. 1B).

Rheumatoid arthritis synovial membrane cells were also cultured in the presence of media alone or media containing 10 μM, 100 μM ketanserine tartrate and levels of TNF were measured. TNF activity was dose dependently inhibited in cells treated with ketanserine tartrate (see FIG. 1C).

These results illustrate that mianserine hydrochloride inhibits the spontaneous release of TNF and from rheumatoid synovial membrane cultures. In addition, these results show that ketanserine tartrate and promazine hydrochloride can also inhibit spontaneous TNF production in rheumatoid synovial membrane cultures.

### Example 2

The murine CIA model has been shown to be clinically similar to human rheumatoid arthritis with a comparable synovitis, bone erosion and panus formation.

Male DBA mice (8-10 weeks of age) were immunized at the base of the tail intradermally with 200 μg type II collagen emulsified in CFA (Difco Laboratories, West Molesey, U.K.). The onset of arthritis was considered to be the day that erythema and/or swelling were first observed, and arthritic mice were given a daily clinical score per limb from 0 to 3, with 0=normal, 1=slight erythema and swelling, 2=pronounced edematous swelling and 3=joint deformity with ankylosis, resulting in a maximum score of 12 per animal. Mianserine treatment started on the day of onset and was administered daily for 7 days. Paw swelling was assessed by measuring hind paw thickness using calipers.

The experiment was started at the onset of arthritis (day 1). Mice were given an interperitoneal injection of vehicle (PBS), 4 mg/kg or 10 mg/kg of mianserine hydrochloride once a day for 7 days. Mice were assessed for (FIG. 2A) clinical score and (FIG. 2B) paw swelling on a daily basis.

Mice treated with mianserine hydrochloride at the day of onset of arthritis (day 1) exhibited an attenuated disease progression over a 7 day period. Disease pathogenesis was assessed by measuring clinical score. The mice treated with mianserine hydrochloride showed a significant improvement to the control group from day 3 onwards (FIG. 2, Graph A, B). This effect of mianserine hydrochloride was dose dependent as 4 mg/kg did not produce as great an effect as the 10 mg/kg group. This was also reflected in the measurement of hind paw swelling that also showed a significant improvement throughout the study between the two groups for the 10 mg/kg group whilst the 4 mg/kg group showed no significant improvement (FIG. 2, Graph B). Histology samples taken from the hind paws of mice treated with mianserine hydrochloride showed a clear reduction in both bone destruction and cell infiltration into the joint (FIG. 2, Graph D) confirming the histology scoring from pooled data (FIG. 2. Graph C) which shows an improvement in the score at 4 mg/kg mianserine hydrochloride group with a significant effect measured in the 10 mg/kg group.

These results illustrate that mianserine hydrochloride inhibits disease progression in the CIA model of rheumatoid arthritis.

### Example 3

Primary human macrophages, murine RAW 264.7 cells and synovial fibroblasts were incubated with 30 μg/ml mianserine hydrochloride for 30 minutes and then stimulated for 6 hours with TLR ligands (R-848, Pam3, LPS, flagellin, malp-2, poly IC or CpG). Human macrophages showed a decreased production of TNF in response to R-848 but not Pam3, LPS, flagellin or Malp-2 in the presence of mianserine hydrochloride (FIG. 3. Graph A).

Murine RAW 264.7 cells were used to examine CpG induced TNF, as unlike human macrophages they respond to CpG, a TLR9 ligand. Mianserine hydrochloride was able to inhibit TNF production in these cells on stimulation with CPG (FIG. 3, Graph B).

To measure activation of TLR3, synovial fibroblasts were infected with a NF-κB reporter virus. Cells were stimulated with LPS as a control and poly IC to activate TLR3. Mianserine hydrochloride was able to significantly inhibit TLR3 induced NF-κB activation but had no effect on LPS induced NF-κB activation (FIG. 3, Graph C).

This shows that signaling from toll-like receptors (TLRs) 3, 8 and 9 are selectively inhibited by mianserine hydrochloride and that it does not have general anti-inflammatory properties as it unable to inhibit TNF stimulated by activation of the other TLRs.

### Example 4

Inhibition of TLRs 3 and 8 by mianserine hydrochloride was also observed for other serotonin receptor antagonists. Promazine hydrochloride decreased TNF production in human macrophages stimulated by the TLR7/8 ligand R-848 (FIG. 4, Graph A). FIG. 4B illustrates that promazine hydrochloride, like mianserine hydrochloride inhibits TLR3 induced signalling in human macrophages (FIG. 4, Graph B).

### Example 5

TLR3, 7, 8 and 9 are expressed in human RA synovial tissue. mRNA from synovial tissue was extracted, DNAase treated and then analysed by RT-PCR. Expression of message for TLR3, 7, 8 and 9 was detected by RT-PCR from the mixed population of cells in human rheumatoid synovial tissue in three unrelated donors 1-3 (FIG. 5, Graph A).

TLR3, 8 and 9 were further investigated using FACS. Cells were washed, then blocked with 10% human serum in PBS containing 0.01% azide for 30 minutes at 4° C. or for intracellular staining cells were fixed in 2% paraformaldehyde and permeabilised with 0.1% saponin (Sigma, USA) before blocking. Cells were then incubated with FITC conjugated α-TLR3, α-TLR8, α-TLR9 or isotype controls for 1 hr at 4° C. and then washed before analysis on a Becton-Dickinson LSR flow cytometer. A directly conjugated antibody was not available to examine TLR7 expression.

TLR3, 8 and 9 protein expression was detected at low levels on the cell surface but was more readily detected upon intracellular staining (FIG. 5, Graph B).

To check if these TLRs were functional in synovial membrane cultures, cells were stimulated with the appropriate TLR ligands at the time of seeding the cells (FIG. 5, Graph C). These cells spontaneously release cytokines into the media. TNF production was increased on stimulation of cells with Poly IC for TLR3 (334%±60 P=0.0002, n=12) and R-848 for TLR7/8 (515%±76 P<0.0001, n=11), but imiquimod, a TLR 7 ligand and CpG a TLR9 ligand (see FIG. 5, Graph C) were unable to stimulate further release of cytokines over the spontaneous levels. This demonstrates that TLR 3 and 8 are both functional in rheumatoid synovial membrane cultures.

In summary, these results shows that TLRs 3, 7, 8 and 9 are expressed in rheumatoid membrane synovial cells but only TLRs 3 and 8 functionally induce increased TNF production in rheumatoid membrane synovial cells.

### Example 6

We have shown that mianserine hydrochloride and promazine hydrochloride both suppress TNF production from human rheumatoid synovial membrane cultures, and show inhibition of some of the TLRs. TLR3, 7, 8 and 9 are classically viewed as having an endosomal localization. Chloroquine has been reported to inhibit the function of TLR3, 7, 8 and 9, as it increases the pH inside vesicles, which is essential for functional signaling from TLR3, 7, 8 and 9. To confirm that inhibition of cytokine release from RA cultures by mianserine hydrochloride and promazine hydrochloride may be through their ability to inhibit signaling through endosomal TLRs, we also measured cytokine release in cells incubated with chloroquine. There was a dose dependant effect of chloroquine with the best inhibition being observed at 100 μM, both TNF and IL-1 release were inhibited (FIG. 6).

Thus, chloroquine inhibits spontaneous TNF and IL-1 release from rheumatoid synovial membrane cultures.

### Example 7

Primary human macrophages were incubated with 5 μg/ml of GBR12909, 5 μg/ml of clotrimazole, 5 μg/ml of Propidium iodide (PI), 30 μg/ml mianserine hydrochloride and 30 μg/ml promazine hydrochloride for 30 minutes and then stimulated for 6 hours with the TLR4 ligand LPS (FIG. 7A), with R848 a TLR7/8 ligand (FIG. 7, Graph B), or with poly IC a TLR3 ligand (FIG. 7C). Supernatants were collected and measured for TNF or IP-10. None of the drugs tested were able to inhibit LPS induced TNF (FIG. 7, Graph A), only the serotonin receptor antagonists (mianserine hydrochloride and promazine hydrochloride) were able to suppress R-848 induced TNF production (FIG. 7, Graph B). All the drugs were able to inhibit IP-10 production induced by Poly IC (FIG. 7, Graph C).

The culture of rheumatoid arthritis membrane cells was as described above for Example 1. In the experiment, rheumatoid arthritis synovial membrane cells were cultured for 24 hours in the presence of media alone or media containing 5 μg/ml of GBR12909, 5 μg/ml of clotrimazole, 5 μg/ml of Propidium iodide (PI) and 504 ml mianserine hydrochloride. Supernatants were harvested and levels of the cytokine TNF were measured by ELISA. Addition of GBR12909, clotrimazole, Propidium iodide at the time of culture did not lead to a significant decrease in the levels of TNF (FIG. 7, Graph D). Mianserine hydrochloride was used as a positive control and was able to decrease the TNF levels in these cultures.

### Example 8

RA synovial membrane cultures were incubated with either 20 μg/ml ondansetron, 5 μg/ml ritanserin, 10 μg/ml metergoline, 20 μg/ml loxapine, 5 μg/ml methiothepin or 30 μg/ml mianserine hydrochloride for 24 hours (FIG. 8). Ondansetron, metergoline, loxapine, ritanserin and methiothepin were purchased from Sigma (USA). TNF production was measured by ELISA.

This shows that the serotonin receptor antagonists; ondansetron, metergoline, loxapine, ritanserin, methiothepin and mianserine hydrochloride are able to inhibit spontaneous TNF production from human RA synovial membrane cultures.

### Example 9

Primary human macrophages were incubated with media containing 0.3, 1, 10 or 20 μg/ml R-848 with media alone or in the presence of either (A) 2.5, 5 or 10 μg/ml mianserine or (B) 5 μg/ml mianserine for 6 hours. TNF production was measured by ELISA.

This shows that mianserine hydrochloride competitively inhibits the production of TNF induced by R-848 (FIG. 9). As the concentration of R-848 increases, higher concentrations of mianserine hydrochloride are required to achieve a 50% inhibition of the control TNF production levels (see FIG. 9(A)). The inhibitory effect of mianserine hydrochloride is diminished with increasing concentrations of R-848 (see FIG. 9(B)).

Our studies suggest mianserine hydrochloride is a competitive inhibitor of R-848.

### Example 10

(A) Primary human macrophages were incubated with media containing 5, 10 or 20 μg/ml ondansetron for 30 minutes prior to stimulation with 1 μg/ml R-848 for 6 hours. TNF production was measures by ELISA. The results are illustrated in FIG. 10(A).

(B) Primary human macrophages were incubated with media containing 30 μg/ml ondansetron for 30 minutes prior to stimulation with 1 μg/ml R-848 or 10 ng/ml LPS for 6 hours (n=3). Data is shown as a percentage of the maximal response for R-848 or LPS and was pooled from three separate donors. The results are illustrated in FIG. 10(B) *=p<0.05.

FIG. 10(C) illustrates inhibition of TNF from RA synovial membrane cultures incubated with 20 μg/ml ondansetron for 24 hours (n=3), *=p<0.05.

This shows that ondansetron inhibits the production of TNF induced by R-848 (FIG. 10) and the spontaneous TNF production from human RA synovial cultures.

### Example 11

Primary human macrophages were pre-incubated with 10 or 100 μM serotonin (5-HT) for 30 minute and then stimulated with 1 μg/ml R-848 for 6 hours (FIG. 11). TNF production was measured by ELISA. Serotonin (5-HT) was purchased from Sigma (USA)

This shows that signaling from toll-like receptors TLR8 is not inhibited by serotonin (5-HT) and that the ability of the 5-HT receptor antagonists to inhibit TNF production induced by TLR8 is not due to effects of serotonin.

### Example 12

Primary human macrophages were pre-incubated with 30 μg/ml ondansetron, 10 μg/ml metergoline, 20 μg/ml loxapine, 5 μg/ml ritanserin or 5 μg/ml methiothepin for 30 minutes and then stimulated with 1 μg/ml R-848 for 6 hours (FIG. 12). TNF production was measured by ELISA. Ondansetron, metergoline, loxapine, ritanserin and methiothepin were purchased from Sigma (USA).

This shows that Ondansetron, metergoline, loxapine, ritanserin and methiothepin are able to inhibit R-848 induced TNF induction.

### Example 13

RA synovial membrane cultures were incubated with either media alone or media containing 5, 10 or 20 μg/ml simvastatin, 20 μg/ml mianserine or as combinations of mianserine and simvastatin for 24 hours (FIG. 13). TNF production from triplicate cultures was measured by ELISA. Simvastatin was purchased from Sigma (USA).

This shows that simvastatin and mianserine are able to inhibit spontaneous TNF production from human RA synovial membrane cultures and produce an additive effect on the inhibition of TNF when incubated together.

1.-6. (canceled)

7. A kit comprising a pharmaceutical composition comprising a serotonin receptor antagonist in an amount effective for treating inflammatory arthritis or a related condition and instructions indicating that the composition is for use in treating inflammatory arthritis or a related condition.

8. The kit of claim 7, wherein the serotonin receptor antagonist is mianserine, promazine, ketanserine, ondansetron, metergoline, loxapine, ritanserin or methiothepin.

9. The kit according to claim 7, wherein the inflammatory arthritis is rheumatoid arthritis.

10. The kit according to claim 7 wherein the serotonin receptor antagonist is administered in combination with a further anti-inflammatory agent.

11. The kit according to claim 10, wherein the further anti-inflammatory agent is a non-steroidal anti-inflammatory agent (NSAID), a statin, a disease modifying anti-rheumatic drug (DMARD), a biological agent, a steroid, an immunosuppressive agent, a salicylate and/or a microbicidal agent.

12. A method of treating inflammatory arthritis or a related condition in a subject, comprising administration of a serotonin receptor antagonist to the subject.

13. The method of claim 12, wherein the serotonin receptor antagonist is mianserine, promazine, ketanserine, ondansetron, metergoline, loxapine, ritanserin or methiothepin.

14. The method of claim 12 wherein the subject is a mammal.

15. The method of claim 14, wherein the mammal is a human.

16. The method of claim 12, wherein the inflammatory arthritis is rheumatoid arthritis.

17. The method according to claim 12, wherein the serotonin receptor antagonist is administered in combination with a further anti-inflammatory agent.

18. The method according to claim 17, wherein the further anti-inflammatory agent is a non-steroidal anti-inflammatory agent (NSAID), a statin, a disease modifying anti-rheumatic drug (DMARD), a biological agent, a steroid, an immunosuppressive agent, a salicylate and/or a microbicidal agent.

19. The method according to claim 12, wherein the related condition is psoriatic arthritis, juvenile arthritis, ankylosing spondelytis, Crohn's disease or psoriasis.
